(19) United States
(12) Patent Application Publication
(10) Pub. No.: US 2025/0276123 A1
(43) Pub. Date: Sep. 4, 2025

(54) SUCTION TUBE ADAPTER

(71) Applicant: The Feinstein Institutes for Medical Research, Manhasset, NY (US)

(72) Inventors: Jonathan Robert Danoff, Great Neck, NY (US); Todd Goldstein, Manhasset, NY (US); Allison Neuwirth, Long Beach, NY (US)

(21) Appl. No.: 19/068,109

(22) Filed: Mar. 3, 2025

Related U.S. Application Data
(60) Provisional application No. 63/560,995, filed on Mar. 4, 2024.

(51) Int. Cl. A61M 1/00; A61M 39/10; A61M 39/12

(52) U.S. Cl. CPC A61M 1/79; A61M 39/105; A61M 39/12; A61M 2039/1077

(57) ABSTRACT

An adapter for reducing or eliminating clogs during a surgical procedure connects a suction tube with a collection canister or aspirator mechanism. An inlet portion at the distal end of the adapter defines a central channel. The inlet portion has an outer surface that engages with an inner surface of the end of the suction tube. The diameter of the central channel is nearly equal to the inner diameter of the suction tube to form a continuous or nearly continuous inner surface. An outlet portion at proximal end of the adapter forms an interference fit with an outer surface of an inlet port of the collection canister. A shoulder within the adapter at a proximal end of the central channel contacts the end surface of the port to form a continuous or nearly continuous inner surface between the central channel and the port. The adapter eliminates gaps where the tube is connected with the port that might collect debris, such as bone fragments, and prevents the debris from collecting and forming a clog.

### REFERENCE TO RELATED APPLICATIONS

This application claims priority under 35 U.S.C. § 119 to U.S. Provisional Patent Application No. 63/560,995, filed on Mar. 4, 2024. The disclosure of that application is incorporated herein by reference.

### BACKGROUND

### Field

This disclosure relates to a device for connecting a suction tube to an aspiration device for removing materials from a surgical field. More particularly, the disclosure relates to a device that provides an improved connection between a suction tube to a suction canister or other device for removing fluids and tissues and debris from a surgical area and that smoothly changes the cross section of the connection to reduce clogging of fluids or tissues.

Surgical procedures often require that tissues and fluids be removed from the surgical incision so that the surgeon can visualize the surgical field. Blood vessels and organs that are dissected during the procedure will leak fluids into the surgical field. Irrigation of the surgical field will also cause fluid to collect. Tissue particles released during a procedure may also collect in the surgical field. In some surgeries, such as orthopedic surgery, this tissue may include bone fragments.

Tissue and fluids are commonly removed using a suction tube connected with a collection device and a source of vacuum. During a procedure, a practitioner, or in the case of a robotically assisted surgery, a medical robot, manipulates the distal end of the suction tube to collect and remove tissues and fluids that might obscure the view of the surgical field. Vacuum draws the tissue and fluids through the tube and into a canister where it is collected for disposal.

Surgical field suction is used during orthopedic surgery to remove blood, fat, debris, and other fluids from the surgical field and wound. During joint replacement surgery, for example, bone debris is pulled by negative pressure into the suction tubing which carries it to a canister connected with a suction machine where it is collected for disposal. Under normal flow conditions the debris can flow through the tube without interruption. However, at the connection between the tube and the suction machine or the canister, there may be a pressure differential as well as a step down in inner diameter tube size. This can lead to the debris lodging itself in the tube leading to a decrease in flow rate or complete blockage of flow in the tube. The problem is addressed by cleaning out this clog or replacing the suction tubes and canister, stopping surgery for 1-5 minutes per blockage.

### SUMMARY

The present disclosure relates to a device for connecting a suction tube with a collection device that overcomes these problems.

According to one aspect, there is provided a suction tube adapter that provides a consistent flow of debris from the suction tubing to the suction machine. The adapter eliminates or removes sudden changes in inner diameter of the path from the suction tubing into suction device, consequentially eliminating the sudden decrease in suction pressure at this junction.

According to one embodiment, the adapter has an inflow end that connects with a proximal end of the suction tubing, for example, by press fitting the adapter into the bore of the tubing or into a fitting at the end of the suction tube. The outflow end of the adapter is press-fit over the end port of a suction machine cannister. The inflow end is shaped so that a distal-most end is flush, or nearly flush with the inside surface of the end of the suction tube. At the outflow end, the inner cross section includes a step shaped to join with a connection on the canister to create a connection with a constant or smoothly varying cross section. By eliminating or reducing sudden changes in diameter or cross section, debris that might become trapped are allowed to flow smoothly from the suction tube into the canister.

According to another embodiment, an adapter is provided with an outflow end, such as in the previous embodiment, that connects with the end port of the cannister to provide a smooth transition. According to this embodiment, at the inflow end of the adapter, a locking mechanism is provided to connect the proximal end of the suction tube with the adapter. The locking mechanism includes two or more tabs extending distally from the adapter. The tabs define an opening sized to accept insertion of the proximal end of the suction tube such that, when it is inserted, the inner bore of the suction tube abuts the inflow end of the adapter. According to one aspect, the inner bore of the adapter is the same diameter as the inner bore of the end portion of the suction tube so that, when the suction tube abuts the inflow end, a smooth transition is provided between the suction tube and the adapter. According to one aspect, the tabs are flexible and can be bent inward, toward the space for receiving the suction tube. A locking ring is provided to encircle the tabs. The inside surface of the ring and the outside surface of the tabs are formed so that, when the ring is moved in the proximal direction, the ring presses the tabs inward. The tabs press against the outside surface of the suction tube, fixing the proximal end of the tube against the inflow end of the adapter. This can allow tubes of varying sizes to mate with a single adapter.

The adapter can be used with a ‘trap’ to remove bone and other debris, with a ‘filter’ to capture and separate the debris, or in full continuity with the suction tubing. Currently, it is used in continuity with the current suction tubing in serial fashion. The adapter can be used with various sized suction tubing making this solution universal for all tube sizes and shapes. It is also agnostic to the brand of suction tubing and particular suction machine being used.

Through intraoperative testing it has been found that various embodiments of the adapter within the scope of the disclosure had more than 98% success at avoiding clogs during orthopedic surgery procedures.

According to one embodiment, an adapter provides for a smooth transition between the proximal end of various brands of suction tubing with commercial aspiration systems, such as the Neptune 3 Waste Management System manufactured by Stryker. It was found that, without an adapter according to embodiments of the disclosure, bone fragments from orthopedic surgeries can get stuck on the “shelf” where the tubing meets the inlet of the machine. An adapter according to the disclosure provides a convenient, low-cost device that allows for the waste material to travel into the machine without clogging.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a perspective view of a cannister for collecting material aspirated during a surgical procedure connected with a suction tube and a source of vacuum;

FIG. 2 is a perspective view of a proximal end of a suction tube that is connected with the cannister of FIG. 1;

FIG. 3 is a perspective view of the cannister of FIG. 1;

FIG. 4 is a cross section of a suction tube connected with the cannister of FIG. 1 according to the prior art;

FIGS. 5A, 5B, and 5C are a perspective view, an elevation view, and a cross-section view, respectively, of an adapter according to an embodiment of the disclosure;

FIG. 6 is a cross-section view of the adapter of FIGS. 5A-5C connected with the suction tubing and cannister of FIGS. 1-3;

FIG. 7A shows the adapter of FIGS. 5A-5C connected with the end of the suction tube of FIG. 2 and FIG. 7B shows the suction tube and adapter connected with the port of an aspiration mechanism;

FIGS. 8A and 8B are perspective views of an adapter according to a further embodiment of the disclosure;

FIG. 9 is an axial view of the distal end of the adapter of FIGS. 8A and 8B;

FIGS. 10 and 11 are a cross-section view and a perspective view, respectively of the adapter of FIGS. 8A and 8B connecting a suction tube with a canister according to an embodiment of the disclosure;

FIG. 12 is a cross-section view of an adapter according to another embodiment of the disclosure; and

FIG. 13 shows a cross-section view of an adapter according to another embodiment of the disclosure.

### DETAILED DESCRIPTION

For purposes of this disclosure, the terms “distal,” “distally,” “distal of” and the like will be used throughout this disclosure to refer to the direction or relative position away from the operator of the device and toward the body of a patient being treated using the device. The terms “proximal,” “proximally,” “proximal of” and the like will be used throughout this disclosure to refer to the direction toward the operator of the device and away from the body of a patient being treated using the device.

Embodiments are described in terms of treatment of a human patient. The disclosure is not limited to devices to treat humans and is applicable to perform veterinary procedures on animals.

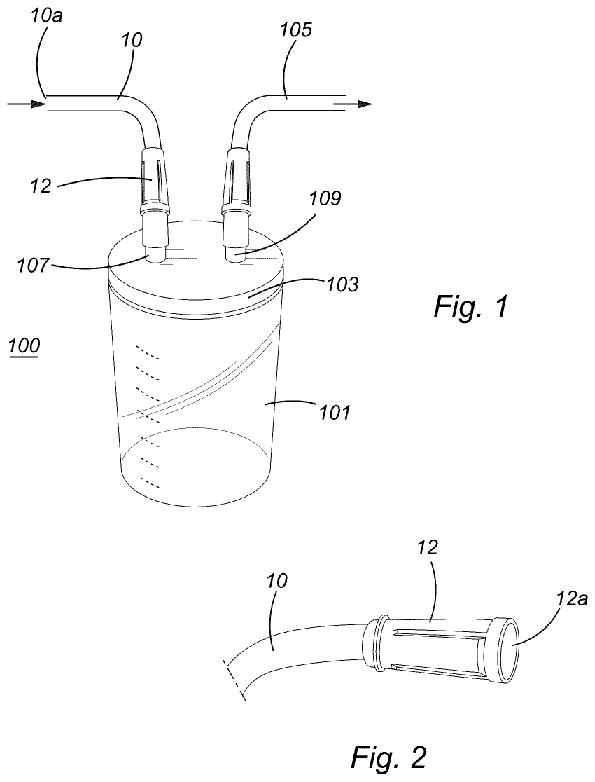

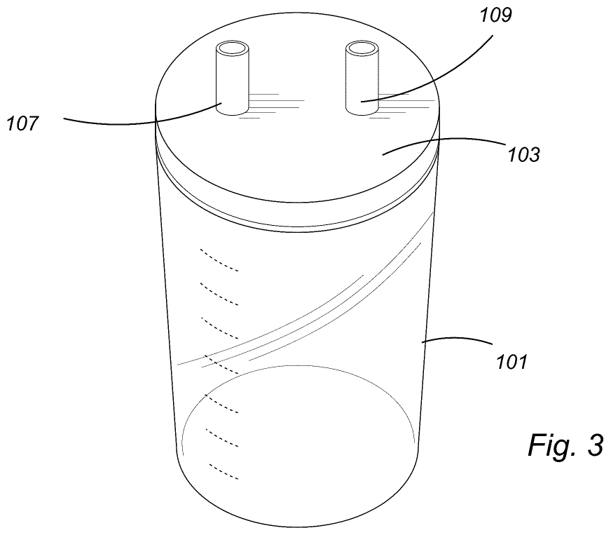

FIGS. 1 to 4 show components of a surgical aspiration system 100. Suction tube 10 includes a tip 10a. Tip 10a is adapted to collect tissue, fluids and debris from a surgical field. Tube 10 is sufficiently long to allow tip 10a to access the necessary portions of the surgical field and to carry materials to a collection canister 101. End portion 12 of tube 10 connects with inlet port 107 extending from lid 103 of canister 101. Collected materials flow through port 107 and into the canister for disposal. End portion 12 may be adapted to serve as a coupling, allowing lengths of tubing 10 to be joined end to end. As shown in FIG. 2, end portion 12 includes an opening 12a sized to form an interference fit with inflow port 107.

As shown in FIG. 1, outlet port 109 extends from lid 103 of cannister 101 and is connected with vacuum line 105. A source of vacuum pumping is applied via line 105. Air is drawn from canister and from suction tube 10 by the vacuum pump, resulting in negative pressure with cannister 101, drawing the collected materials into the cannister. Lid 103 includes structures (not shown) to prevent the collected materials from being drawn into vacuum line 105.

Vacuum line 105 may be connected with a vacuum source such as bedside aspirator or to a facility-wide vacuum system. According to some embodiments, instead of applying vacuum via line 105, cannister 101 connects directly with suction apparatus, such as a surgical aspirator or medical waste management system.

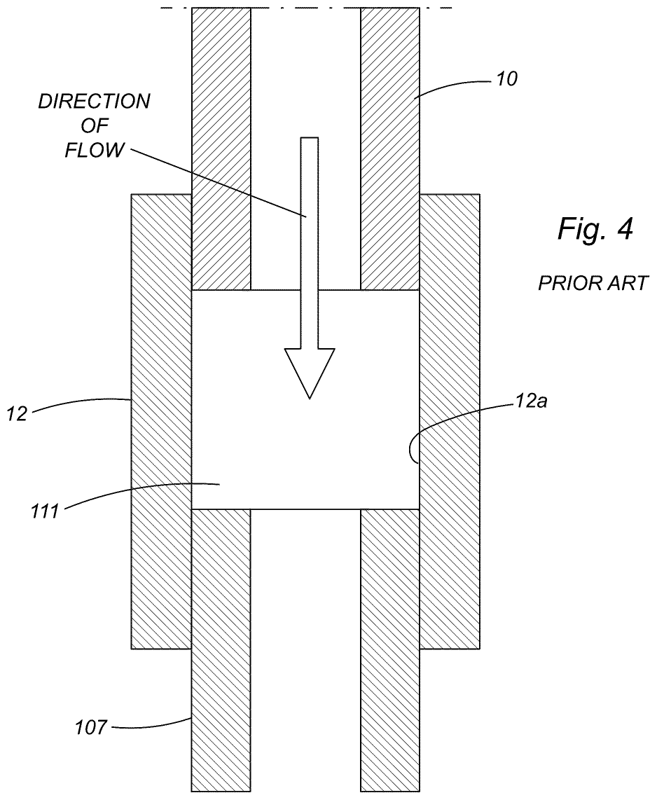

FIG. 4 shows a cross section of tubing 10 connected with inflow port 107 by end portion 12 using known apparatus. Material collected by tip 10a flows in the direction of flow from tube 10, through end portion 12 and into inflow port 107 to be collected by cannister 101. The inner diameter of end portion 12 is larger than the inner diameter of inflow port 107, leaving a gap region 111.

Gap region 111 may result in clogging of port 107. Where the material aspirated from the surgical field includes hard materials such as bone fragments generated during orthopedic surgery, these hard materials can become lodged in gap 111, trapping materials and fluids leading to a clog. This may require the surgeon to stop the medical procedure while the clog is cleared.

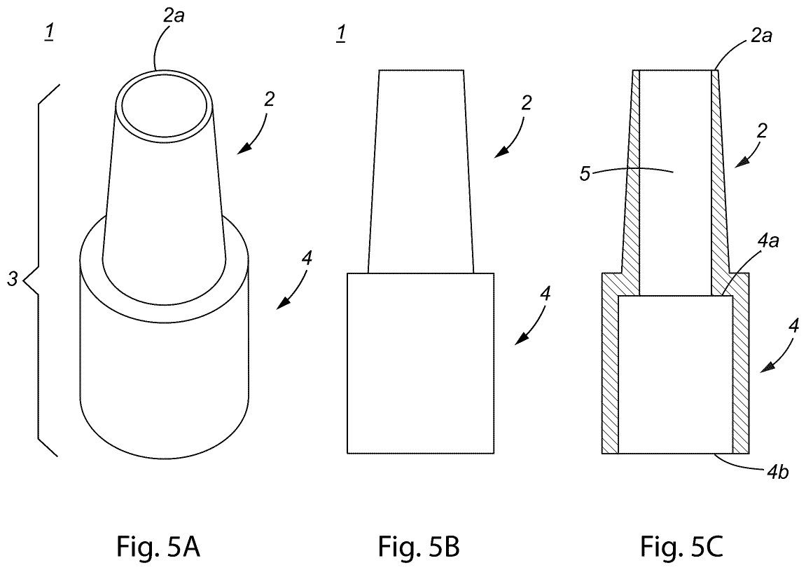

FIGS. 5A, 5B, and 5C show an adapter 1 according to an embodiment of the disclosure. Adapter 1 is formed by adapter body 3 with distal inlet portion 2 and proximal outlet portion 4. Inlet portion 2 has a tapered outer diameter with an edge 2a at its distal end. FIG. 5c shows a cross section of adapter 1 showing the tapered outer surface of inlet portion 2.

According to one embodiment, the inner surface of inlet portion 2 forms a central passage 5 that may be the same as, or slightly larger than the inner diameter of suction tube 10 and/or end portion 12. When assembled with an end portion 12 of suction tube 10, a narrow distal edge 2a lies along the inside surface of end portion 12. Thus, the inner diameter of passage 5 and the inner diameter of end portion 12 are nearly equal. According to one embodiment, the radial width of narrow edge 2a is between about 0 millimeters (“mm”) and about 2 mm. According to a preferred embodiment, narrow edge 2a is less than about 1 mm. According to some embodiments, the width of narrow edge 2a is selected to avoid creating an abrupt change in diameter, which may cause aspirated materials to become trapped.

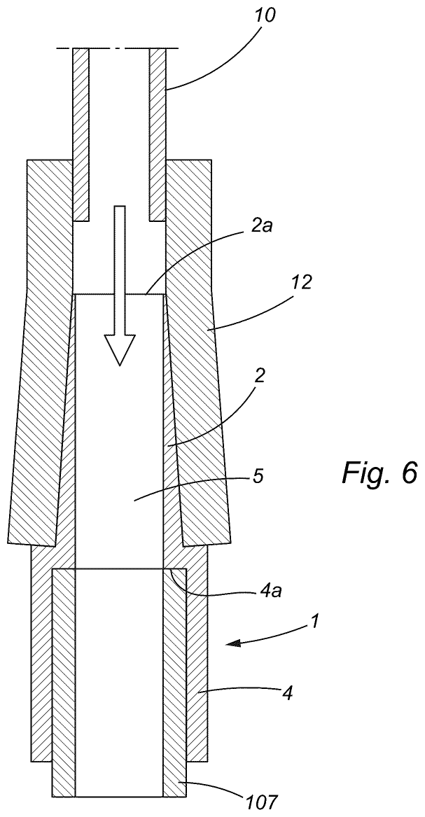

According to one embodiment, outlet portion 4 has a constant inner diameter that is sized to form an interference fit with inlet port 107. FIG. 6 shows a cross section of adapter 1 connected with suction tube 10 and input port 107 of cannister 101. Outlet portion 4 includes shoulder 4a. According to one embodiment, shoulder 4a is shaped to receive the distal end of input port 107.

According to one embodiment, central passage 5 has approximately the same diameter as the inner diameter of port 107 so that a smooth transition is provided between adapter 1 and input port 107. According to another embodiment, the inside of passage 5 can be smooth. According to a further embodiment, passage 5 includes a 3-dimensional surface features 8, as shown in FIG. 13 such as a spiral or straight or undulating grooves, or continuous or interrupted lines along its surface that are adapted to allow the passage of air or liquid to relieve negative pressure or allow liquified material to pass in the event that the main housing becomes clogged or disrupted. According to one embodiment, output portion 4 is sized to form an interference fit with port 107.

As shown in FIG. 6, input portion 2 is engaged with end portion 12 of suction tube 10. The tapered outer surface of inlet portion 2 forms an interference fit with end portion 12. Narrow distal end 2a has a diameter that is the same or nearly the same as the inside diameter of end portion 12. As a result, central channel 5 is equal or nearly equal to an inner diameter of end portion 12 to form a continuous or nearly continuous inner surface. The inside diameter through which aspirated materials flow remains constant, thereby reducing or eliminating the tendance for materials to collect and clog the flow.

According to one embodiment, the diameter of central passage 5 is the same as the inner diameter of port 107 so that there is little or no change in inner diameter from end portion 12 through port 107. According to one embodiment, the difference in diameters of the central passage and the end port is less than about 1 mm.

According to another embodiment, central passage 5 is narrower than the inner diameter of port 107 so that the cross-sectional area through which aspirated material flows gets larger as the material enters port 107. According to this embodiment, clogging of material is avoided because, once the material passes from passageway 5, there is no further reduction in diameter that might cause a gap, such as gap 111 shown in FIG. 4.

According to another embodiment, central passage 5 varies in diameter along its length. An adapter 1 according to this embodiment can form an interface between aspiration tubes 10 and ports 107 made by different manufacturers or of different sizes. According to one embodiment, a kit of adapters 1 is provided to allow aspiration mechanisms with different inlet ports to connect with aspiration tubes with different end portions. The change in diameter along the length of the adapter may be smooth, to avoid an abrupt change in diameter that might trap materials or otherwise clog.

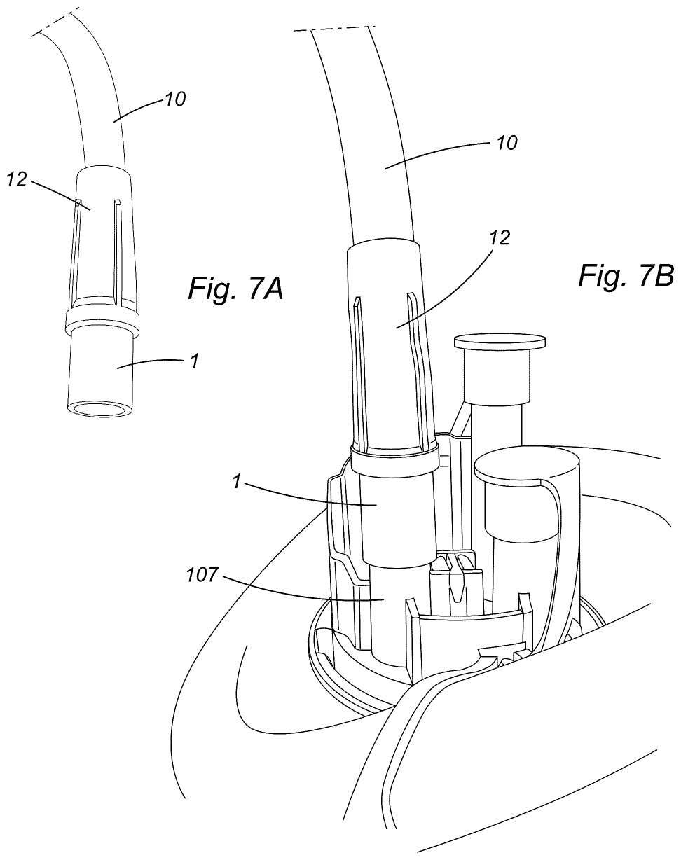

FIG. 7A shows adapter 1 connected with aspiration tube 10 and end portion 12. As shown in FIG. 7B, adapter 1 connects with port 107 of an aspiration mechanism to form a passage from tube 10 into a cannister 101 or other collection device with a continuous or smoothly varying inner diameter.

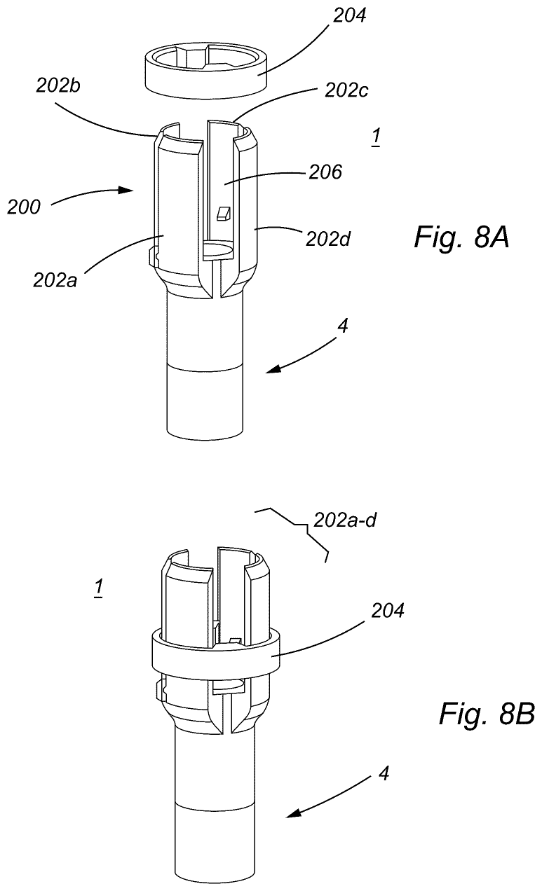

FIGS. 8A and 8B show an adapter 1 according to another embodiment of the disclosure. Proximal outlet portion 4 of adapter 1 is shaped like the outlet portion of the previous embodiments. According to one embodiment, portion 4 forms an interference fit with an input port 107 of a collection cannister 101. Shoulder 4a, such as the one shown in FIG. 5C, may be provided so that the inner diameter of adapter 1 connects with port 107 without a gap or other abrupt change in diameter. Distal inlet portion 200 is formed by a plurality of tabs 202a, 202b, 202c, 202d. Tabs 202a-d are flexible and can be moved inward toward the center of adapter 1. Locking ring 204 fits over tabs 202a-d to flex the tabs inward and to hold them in an inwardly flexed configuration. Four tabs 202a-d are shown, but a fewer or greater number of tabs can be provided with the scope of the disclosure. That is, adapter 1 could have two, three, four, or more tabs within the scope of the disclosure.

Tabs 202a-d define a coupling portion 203. When locking ring 204 is not engaged with tabs 202a-d, as shown in FIG. 8A, coupling portion 203 is sized to receive an end portion of suction tube 10 According to one embodiment, one or more barbs 206 may be provided on tabs 202a-d.

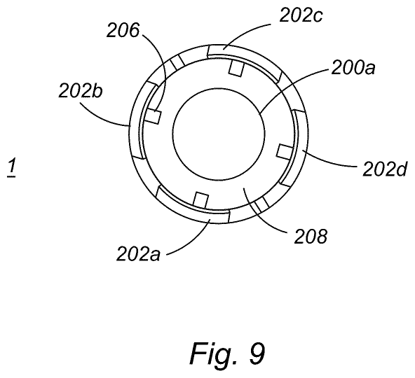

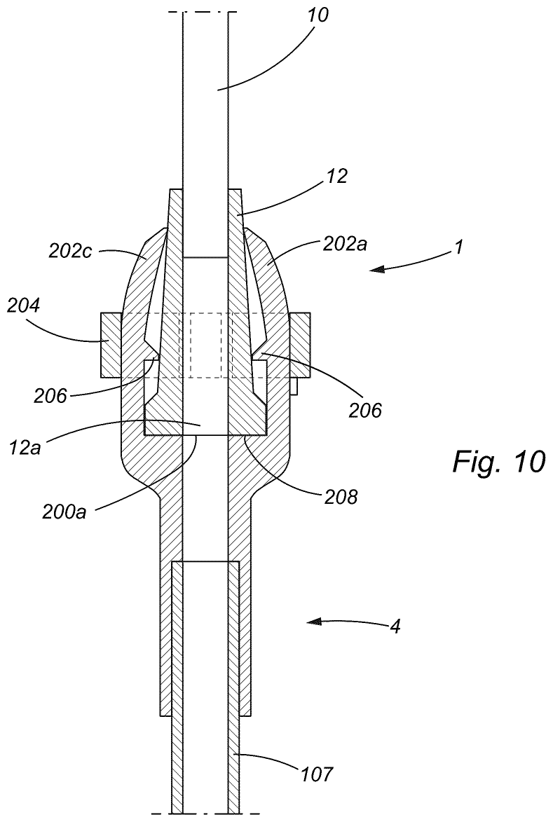

FIG. 9 shows adapter 1 viewed axially along the distal direction. Inner bore 200a is provided through the adapter to connect with port 107. Land 208 surrounds bore 200a. FIG. 10 shows a cross section of adapter 1 connecting suction tubing 10 with port 107. End portion 12 of tubing 10 abuts land 208 surrounding bore 200a. The diameter of bore 200a is the same as the opening 12a at the end of end portion 12 (as shown in FIG. 2) so that little or no gap is formed between the end portion and the inner diameter of adapter 1.

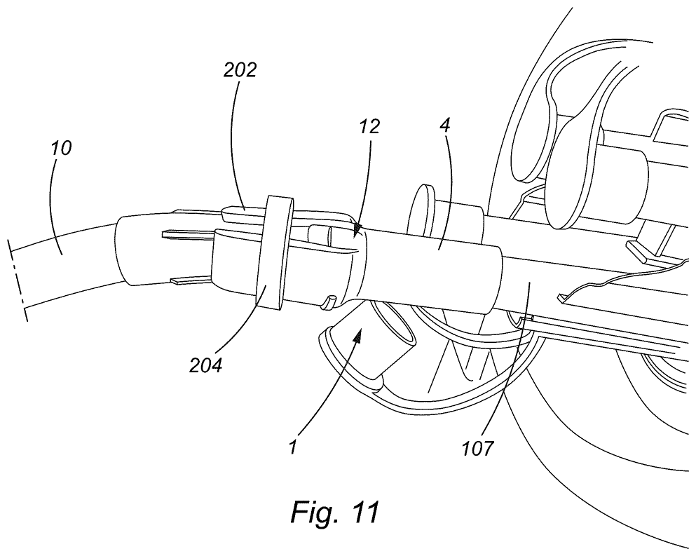

Locking ring 204 is fitted over tabs 202a-d and is moved in the proximal direction to press the tabs against the outside surface of end portion 12 to secure adapter 1 with end portion 12. Barbs 206 are pressed against the outer surface of end portion 12 to further secure adapter 1 with suction tube 10. As with previous embodiments, because the connections between adapter 1, end portion 12, and input port 107 has a diameter with no gaps or other abrupt changes in diameter, the tendency for materials to form a clog is reduced or eliminated. FIG. 11 shows adapter 1 connecting suction tube 10 with port 107.

According to one embodiment, adapter 1, such as disclosed in FIGS. 5A-5C has an inlet portion 2 with a tapered outer surface that is sized to connect with suction tube 10 without an end portion 12. In this embodiment, the proximal end of suction tube 10 is fitted directly over inlet portion 2. According to one embodiment, suction tube 10 is stretched over inlet portion 2 to form a secure, air-tight fit with adapter 1.

When assembled with suction tube 10, the narrow edge 2a lies along the inside surface of suction tube 10. Thus, the inner diameter of passage 5 and the inner diameter of suction tube 10 are nearly equal. According to one embodiment, the radial width of narrow edge 2a is between about 0 millimeters (“mm”) and about 2 mm. According to a preferred embodiment, narrow edge 2a is less than about 1 mm. According to some embodiments, the width of narrow edge 2a is selected to avoid creating an abrupt change in diameter, which may cause aspirated materials to become trapped.

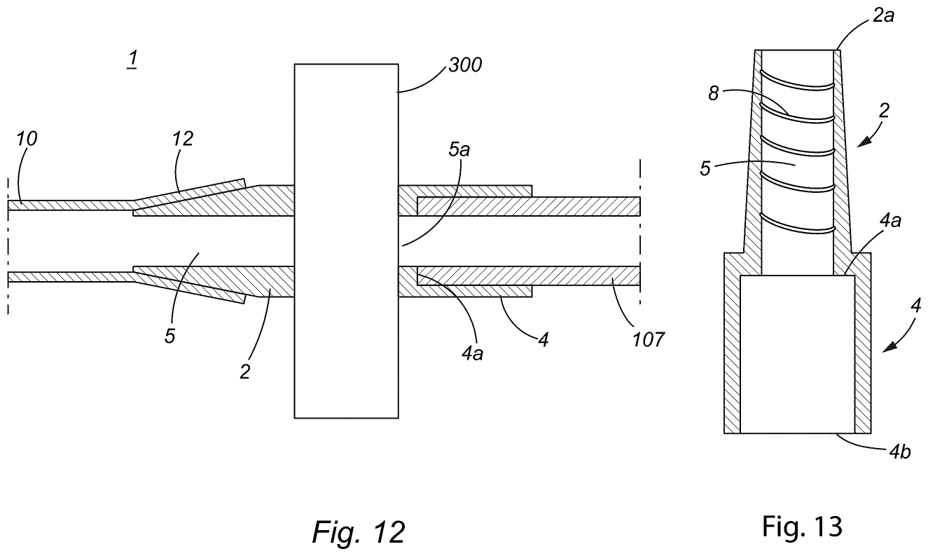

According to some embodiments, adapter 1 can be used with a ‘trap’ to remove bone and other debris or with a ‘filter’ to capture and separate the debris. FIG. 12 shows one embodiment of adapter 1 with trap or filter 300. Inlet portion 2 is shaped to engage with end portion 12 of tube 10 and defines input channel 5 that is continuous or nearly continuous with the inside diameter of end portion 12. Inlet portion 2 connects with the input of trap or filter 300. Outlet portion 4 is connected to the outlet of trap or filter 300. As in previous embodiments, outlet portion 4 is sized to connect with port 107. Outlet portion 4 defines an outlet channel 5a and includes shoulder 4a. Port 107 abuts shoulder 4a so that outlet channel 5a forms a continuous or nearly continuous passageway with port 107.

While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

We claim:

1. An adapter for connecting a suction tube with an aspirator mechanism, the adapter comprising:
a body;
an inlet portion at a distal end of the body defining a central channel and having an outer surface adapted to engage with an inner surface of an end portion of the suction tube, wherein a diameter of the central channel is equal or nearly equal to an inner diameter of the end portion to form a continuous or nearly continuous inner surface therewith;
an outlet portion at a proximal end of the body defining an outlet diameter, wherein the outlet diameter is adapted to form an interference fit with an outer surface of an inlet port of the aspirator mechanism; and
a shoulder at a proximal end of the central channel, the shoulder shaped to contact a distal end surface of the inlet port, wherein the diameter of the central channel is narrower than or equal to the inner diameter of the inlet port.

2. The adapter of claim 1, wherein the diameter of the central channel and the inner diameter of the inlet port are equal or nearly equal.

3. The adapter of claim 1, wherein the inlet portion comprises a tapered outer surface.

4. The adapter of claim 1, wherein the inlet portion comprises a distal edge surrounding the central channel, wherein the distal edge has a radial width of between about 0 mm and about 2 mm.

5. The adapter of claim 4, wherein the radial width is less than about 1 mm.

6. The adapter of claim 1, wherein the diameter of central channel is equal or nearly equal to an inner diameter of the inlet port and wherein contact of the distal end of the inlet port with the shoulder forms a continuous or nearly continuous inner surface between the central channel and an inner surface of the port.

7. The adapter of claim 6, wherein a difference in diameters of the central channel and the inner diameter of the inlet port is less than about 1 mm.

8. The adapter of claim 1, wherein the diameter of central channel is less than an inner diameter of the inlet port.

9. The adapter of claim 1, wherein an inner surface of the central channel comprises a three-dimensional pattern to allow the passage of air or liquid to relieve negative pressure or allow liquified material to pass in the event that the main housing becomes clogged or disrupted.

10. The adapter of claim 9, wherein the three-dimensional pattern comprises a spiral or straight or undulating grooves.

11. The adapter of claim 1, further comprising a trap or a filter disposed along the central channel between the inlet portion and the outlet portion.

12. An adapter for connecting a suction tube with an aspirator mechanism, the adapter comprising:
a body;
an inlet portion at a distal end of the body defining a central channel and having an outer surface adapted to engage with an inner surface of the suction tube, wherein a diameter of the central channel is equal or nearly equal to an inner diameter of the suction tube to form a continuous or nearly continuous inner surface therewith;
an outlet portion at a proximal end of the body defining an outlet diameter, wherein the outlet diameter is adapted to form an interference fit with an outer surface of an inlet port of the aspirator mechanism; and
a shoulder at a proximal end of the central channel, the shoulder shaped to contact a distal end surface of the inlet port, wherein the diameter of the central channel is narrower than or equal to the inner diameter of the inlet port.

13. The adapter of claim 10, wherein the suction tube includes an end portion and wherein the inlet portion engages with the end portion.

14. An adapter for connecting an end portion of a suction tube with an aspirator mechanism, the adapter comprising:
a body;
an inlet portion at a distal end of the body defining a central channel with a channel diameter and comprising two or more tabs, wherein the tabs are spaced apart to define a coupling portion, wherein the end portion fits within the coupling portion;
a locking ring disposed around the outer circumference of the tabs, wherein displacement of the locking ring along the tabs presses the tabs inward against the end portion into a locked configuration
wherein a diameter of the central channel is equal or nearly equal to an inner diameter of the end portion to form a continuous or nearly continuous inner surface therewith;
an outlet portion at a proximal end of the body defining an outlet diameter, wherein the outlet diameter is adapted to form an interference fit with an outer surface of an inlet port of the aspirator mechanism; and
a shoulder at a proximal end of the central channel, the shoulder shaped to contact a distal end surface of the port, wherein the channel diameter is about equal to or less than an inner diameter of the port.
